(19) United States
(12) Patent Application Publication
(10) Pub. No.: US 2025/0186660 A1
(43) Pub. Date: Jun. 12, 2025

(54) CATHETER COATINGS

(71) Applicant: MicroVention, Inc., Aliso Viejo, CA (US)

(72) Inventor: Christopher Ravelo, Aliso Viejo, CA (US)

(21) Appl. No.: 18/972,530

(22) Filed: Dec. 6, 2024

Related U.S. Application Data
(60) Provisional application No. 63/607,787, filed on Dec. 8, 2023.

(51) Int. Cl. A61L 29/08; A61M 25/10

(52) U.S. Cl. CPC A61L 29/085; A61M 25/10

(57) ABSTRACT

A catheter that includes a proximal portion, a distal portion, and an interior lumen therebetween is provided. The distal portion has a distal exterior catheter surface having a coated section that includes a first coating and a second coating. The first coating includes a polymer that includes a D-glucuronic acid monomer and an N-acetyl-D-glucosamine monomer. The second coating includes an ethyl cellulose polymer atop the first coating, and bound to at least a portion of the first coating. The surface area of the first coating is greater than the surface area of the second coating. The second coating masks liquid embolic solutions from the first coating, which helps reduce attractive forces between the liquid embolic solutions and the catheter.

### RELATED APPLICATIONS

This application claims priority to U.S. Provisional Patent Application No. 63/607,787, filed Dec. 8, 2023, the entire content of which is incorporated by reference.

### FIELD

The present disclosure relates to a catheter useful in delivering aqueous liquid embolic solutions to a target site.

### BACKGROUND

Therapeutic embolization is a minimally invasive procedure in which a material is introduced into a blood vessel to produce an occlusion in order to slow or stop blood flow or to fill a space such as an aneurism. This approach has been useful in the treatment of conditions such as gastrointestinal bleeding, arteriovenous malformations, hypervascular tumors, benign growths such as uterine fibroids, and benign prostate hyperplasia (BPH).

Catheter embolization may place medications or synthetic materials called embolic agents through a catheter into a blood vessel to block blood flow to an area of the body. It may be used to control or prevent abnormal bleeding, close off vessels supplying blood to a tumor, eliminate abnormal connections between arteries and veins, or to treat aneurysms. Embolization is a highly effective way to control bleeding and is much less invasive than open surgery.

Despite the benefits afforded by therapeutic embolization, there are difficulties related to the adhesion of water-based liquid embolic solutions on coatings commonly found on catheters. This adhesion may result in destabilization of the embolism and may pose a risk to the patient.

### SUMMARY

In order to solve the above shortcomings, a coating solution was developed that would cover a first coating that is already on the catheter. This second coating would cover the first coating only at the distal portion of the catheter. This second coating would mask the liquid embolic solutions from the first coating, which would help reduce attractive forces between the liquid embolic solutions and the catheter.

### BRIEF DESCRIPTION OF THE DRAWINGS

The following figure is included to illustrate certain aspects of the present disclosure and should not be viewed as exclusive embodiments. The subject matter disclosed is capable of considerable modifications, alterations, combinations, and equivalents in form and function, as will occur to one having ordinary skill in the art and having the benefit of this disclosure.

FIG. 1 shows a catheter (1) with an interior lumen (4), a distal portion (10) and a proximal portion (13). “Distal” represents the insertion portion of the catheter and “proximal” is the portion farthest away from the distal portion.

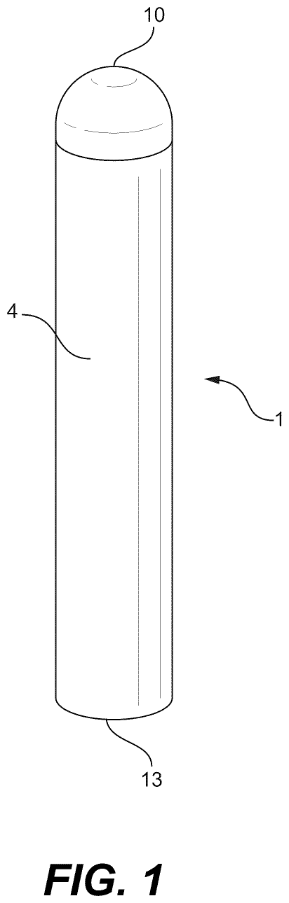

FIG. 2 illustrates a section (11) from the distal end (10), the exterior distal surface is covered by the first coating (22) which extends a distance L1; and a second coating (24) placed atop the first coating which extends a distance L2.

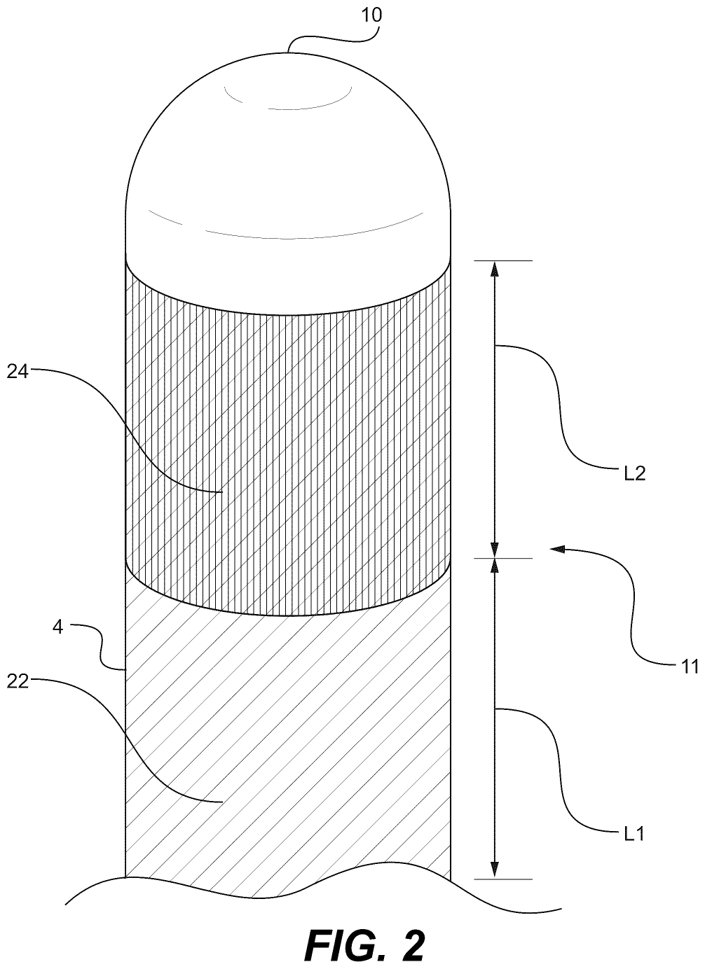

### DETAILED DESCRIPTION

Aspects of the present disclosure are directed to a catheter that includes a proximal portion, a distal portion, and an interior lumen therebetween. The distal portion has a distal exterior catheter surface having a coated section that includes a first coating and a second coating. The first coating includes a polymer that includes a D-glucuronic acid monomer and an N-acetyl-D-glucosamine monomer. The second coating includes an ethyl cellulose polymer atop the first coating, and bound to at least a portion of the first coating. The surface area of the first coating is greater than the surface area of the second coating.

In some embodiments, the first coating extends a distance L1 which is more than about 5.0 cm from the distal portion that is farthest away from the proximal portion. In some embodiments, the first coating extends a distance L1 which is more than about 5.1 cm, more than about 5.2 cm, more than about 5.3 cm, more than about 5.4 cm, more than about 5.5 cm, more than about 5.6 cm, more than about 5.7 cm, more than about 5.9 cm, more than about 6.0 cm, more than about 6.5 cm, more than about 7.0 cm, more than about 7.5 cm, more than about 8.0 cm, more than about 8.5 cm, more than about 9.0, cm more than about 9.5 cm, or more than about 10.0 cm from the distal portion that is farthest away from the proximal portion.

In some embodiments, the second coating extends a distance L2 which is at least about 3.0 cm from the distal portion that is farthest away from the proximal portion and at most about 5.0 cm from the distal portion that is farthest away from the proximal portion. In some embodiments, the second coating extends a distance L2 which is at least about 3.1 cm, at least about 3.2 cm, at least about 3.3 cm, at least about 3.4 cm, at least about 3.5 cm, at least about 3.6 cm, at least about 3.7 cm, at least about 3.8 cm, at least about 3.9 cm, at least about 4.0 cm, at least about 4.1 cm, at least about 4.2 cm, at least about 4.3 cm, at least about 4.4 cm, or at least about 4.5 cm from the distal portion that is farthest away from the proximal portion and at most about 5 cm from the distal portion that is farthest away from the proximal portion.

In some embodiments, L2 is a shorter distance than L1. In some embodiments, the distance L1 is as long as the entire length of the catheter. In other embodiments, the distance L1 is more than the distance L2 and less than the entire length of the catheter. In some embodiments, placing a second coat for a distance L2 on the catheter allows for the catheter to be gripped during medical procedures. In some embodiments, the dual coated length L2 of the catheter has a higher average dynamic friction force compared to the singly coated length L1, which allows for the catheter to be gripped during medical procedures.

In one instance, the average dynamic friction force may be measured by using an Instron 5943 material tester equipped with a 5 N static load cell. A mechanical clamping fixture is attached to the load cell to hold the top of the microcatheter sample as its length is pulled through a hydraulic clamping fixture (clamping force of 1 lb.) submerged in a heated (37° C.) water bath containing distilled water. Each sample can be cycled repeatedly 100 times at a pull rate of e.g., 254 mm/min for 60 mm, with one cycle measured as starting at 0 mm displacement with the hydraulic clamp closed on the sample, then the sample is pulled through the hydraulic clamp for 100 mm displacement, and finally the hydraulic clamp is opened, and the sample returned to 0 mm displacement. The maximum dynamic friction force and the average dynamic friction force at the 30 mm displacement mark can be measured for the dual coated length L2 and singly coated length L1. In some embodiments, the average dynamic friction force in the dual coated length L2, which allows for the catheter to be gripped during medical procedures, is greater than about 100 [gf] at 30 mm displacement for 100 cycles. In some embodiments, the average dynamic friction force in the singly coated length L1 is below about 50 [gf], about 40 [gf], about 30 [gf], or about 20 [gf] at 30 mm displacement for 100 cycles.

In some embodiments, the second coating includes an ethyl cellulose polymer.

In some embodiments, the second coating includes one or more of the following polymers: ethyl cellulose, methyl cellulose, hydroxypropyl cellulose, hydroxyethyl cellulose and hydroxypropylmethyl cellulose.

In some embodiments, the polymer that includes a D-glucuronic acid monomer and an N-acetyl-D-glucosamine monomer is a polymer that includes a hyaluronic acid. In some embodiments, the polymer that includes a D-glucuronic acid monomer and an N-acetyl-D-glucosamine monomer is hyaluronic acid.

In some embodiments, the first coating is a hydrophilic polymeric coating.

In some embodiments, the first coating is a lubricious coating having a higher lubricity compared to an uncoated surface of the catheter.

In some embodiments, the catheter is a microcatheter or a balloon catheter.

Another aspect of the present disclosure is directed to methods that include flowing an aqueous liquid embolic solution through the catheter of one of the foregoing embodiments.

Another aspect of the present disclosure is directed to methods of delivering an aqueous liquid embolic solution to a target site. The methods include flowing the aqueous liquid embolic solution through the catheter of one of the foregoing embodiments.

Another aspect of the present disclosure is directed to methods of treating a vasculopathy in a subject in need thereof. The methods include administering an aqueous liquid embolic to the subject. The administering includes flowing the aqueous liquid embolic through the catheter of one of the foregoing embodiments.

In some embodiments, the vasculopathy is a pseudoaneurysm.

In some embodiments, the vasculopathy is a dissecting aneurysm.

In some embodiments, the vasculopathy is an aneurysm.

In some embodiments, the aneurysm is a fusiform aneurysm.

In some embodiments, the aneurysm is a saccular aneurysm.

In some embodiments, the aneurysm is an aortic aneurysm, a cerebral aneurysm, a popliteal aneurysm, a femoral aneurysm, a mesenteric aneurysm, or a splenic aneurysm.

In some embodiments, the first coating includes a polymer that includes a D-glucuronic acid monomer or an N-acetyl-D-glucosamine monomer, or includes both monomers.

In some embodiments, the first coating includes a polymer that is a derivative of hyaluronic acid. Examples of these derivatives are disclosed, for example, in U.S. Pat. No. 9,644,040. The N-acetyl group of hyaluronic acid may be removed or substituted with a different acyl functionality. A hyaluronic acid derivative may include repeating units of a disaccharide unit comprising D-glucuronic acid and N-acetylglucosamine moieties. A portion of the N-acetyl groups (NHC(O)CH₃) of the N-acetylglucosamine moiety of the disaccharide moieties may be independently substituted with hydrogen or a group of the formula —N—C(O)—(C₂-C₂₀)-alkyl, —N—C(O)—(C₂-C₂₀)-alkenyl or —N—C(O)—(C₂-C₂₀)-alkynyl. The hyaluronic acid derivative may have a molecular weight of at least about 20 kDa. The hyaluronic acid derivative may be a pharmaceutically acceptable salt, ester, or glucoside thereof.

A coating solution of ethyl cellulose in ethanol at 0.1-1% m/m may be prepared. The solution may be prepared at room temperature, or heated (e.g., in an oven) at 50-60° C. to accelerate dissolution. The catheter's distal portion may be dipped into this coating solution and allowed to dry. The resulting film may mask the liquid embolic from the previous coating (i.e., inner coating), reducing attractive forces between the embolic and the inner coating.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the disclosure (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope of the disclosure otherwise claimed. No language in the specification should be construed as indicating any non-claimed element essential to the practice of the disclosure.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience or patentability. When any such inclusion or deletion occurs, the specification is deemed to contain the group as modified thus fulfilling the written description of all Markush groups used in the appended claims.

Certain embodiments of this disclosure are described herein, including the best mode known to the inventors for carrying out the disclosure. Of course, variations on these described embodiments will become apparent to those of ordinary skill in the art upon reading the foregoing description. The inventor expects skilled artisans to employ such variations as appropriate, and the inventors intend for the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

Furthermore, references to patents and printed publications may have been made in this specification. Each of the above-cited references and printed publications are individually incorporated herein by reference in their entirety.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

We claim:

1. A catheter, comprising:
a proximal portion, a distal portion, and an interior lumen there between;
wherein
the distal portion has a distal exterior catheter surface having a coated section that includes a first coating and a second coating,
the first coating comprises a polymer comprising a D-glucuronic acid monomer and an N-acetyl-D-glucosamine monomer,
the second coating comprises an ethyl cellulose polymer atop the first coating, and bound to at least a portion of the first coating, and
the surface area of the first coating is greater than the surface area of the second coating.

2. The catheter of claim 1, wherein the first coating extends more than about 5.0 cm from the distal portion that is farthest away from the proximal portion.

3. The catheter of claim 1, wherein the second coating extends at least about 3.0 cm from the distal portion that is farthest away from the proximal portion and at most about 5.0 cm from the distal portion that is farthest away from the proximal portion.

4. The catheter of claim 1, wherein the second coating comprises an ethyl cellulose polymer.

5. The catheter of claim 1, wherein the polymer comprising a D-glucuronic acid monomer and an N-acetyl-D-glucosamine monomer is hyaluronic acid.

6. The catheter of claim 1, wherein the first coating is a hydrophilic polymeric coating.

7. The catheter of claim 1, wherein the first coating is a lubricious coating having a higher lubricity compared to an uncoated surface of the catheter.

8. The catheter of claim 1, wherein the catheter is a microcatheter or a balloon catheter.

9. A method, comprising flowing an aqueous liquid embolic solution through the catheter of claim 1.

10. A method of delivering an aqueous liquid embolic solution to a target site, comprising flowing the aqueous liquid embolic solution through the catheter of claim 1.

11. A method of treating a vasculopathy in a subject in need thereof, comprising administering an aqueous liquid embolic to the subject, wherein the administering comprises flowing the aqueous liquid embolic through the catheter of claim 1.

12. The method of claim 11, wherein the vasculopathy is a pseudoaneurysm.

13. The method of claim 11, wherein the vasculopathy is a dissecting aneurysm.

14. The method of claim 11, wherein the vasculopathy is an aneurysm.

15. The method of claim 14, wherein the aneurysm is a fusiform aneurysm.

16. The method of claim 14, wherein the aneurysm is a saccular aneurysm.

17. The method of claim 14, wherein the aneurysm is an aortic aneurysm, a cerebral aneurysm, a popliteal aneurysm, a femoral aneurysm, a mesenteric aneurysm, or a splenic aneurysm.
